(19) United States
(12) Patent Application Publication
(10) Pub. No.: US 2026/0027084 A1
(43) Pub. Date: Jan. 29, 2026

(54) TREATMENT OF MULTIPLE MYELOMA

(71) Applicant: SpringWorks Therapeutics, Inc., Stamford, CT (US)

(72) Inventors: Todd Webster SHEARER, Stamford, CT (US); Rex WILLIAMS, Stamford, CT (US); Mark JOHNSON, Stamford, CT (US)

(21) Appl. No.: 18/993,488

(22) PCT Filed: Jul. 26, 2023
(86) PCT No.: PCT/US2023/028741

§ 371 Date: Jan. 10, 2025

Related U.S. Application Data
(60) Provisional application No. 63/369,733, filed on Jul. 28, 2022.

(51) Int. Cl. A61K 31/417; A61K 40/11; A61K 40/31; A61K 40/42; A61P 35/00; G01N 33/574

(52) U.S. Cl. CPC A61K 31/417; A61K 40/11; A61K 40/31; A61K 40/4215; A61P 35/00; G01N 33/57488

(57) ABSTRACT

The present disclosure provides methods of treating multiple myeloma in a patient in need thereof comprising administering a gamma secretase and a B-cell maturation antigen (BCMA)-directed therapy to the patient.

This application is a national stage of International Patent Application No. PCT/US2023/028741, filed Jul. 26, 2023, which claims the benefit of U.S. Provisional Application No. 63/369,733, filed Jul. 28, 2022, which are hereby incorporated by reference.

### FIELD OF THE INVENTION

The present disclosure provides methods of treating multiple myeloma in a patient in need thereof comprising administering a gamma secretase inhibitor (e.g., nirogacestat) or a pharmaceutically acceptable salt thereof and a B-cell maturation antigen (BCMA)-directed therapy to the patient.

### BACKGROUND

Multiple myeloma is a bone marrow cancer that affects more than 30,000 patients each year in the United States. Multiple myeloma is currently considered an incurable disease, but overall survival has improved significantly with recent technological advancements in genetic research that have allowed researchers to identify novel targets of the disease. There has been specific interest in B-cells because of their role in modulating the immune response to cancer. B-cell maturation antigen (BCMA) plays an important role in B-cell proliferation and survival, and has been a focus of study and treatment for diseases like multiple myeloma. BCMA is expressed on the cell surface of both healthy and cancerous plasma cells and exhibits both intra-and extracellular functional components. The extracellular component of BCMA, membrane bound BCMA (mbBCMA) can be cleaved from the cell surface by γ-secretase to generate soluble BCMA (sBCMA). sBCMA levels have been shown to be correlated with disease progression and prognosis.

Because of these findings, there has been interest in researching and testing BCMA expression levels and its effects on disease. It is standard procedure to analyze BCMA expression on plasma cells using bone marrow samples. However, collecting bone marrow is expensive, invasive, and painful for the patients.

There is a need for improved strategies for treating multiple myeloma and methods for improving existing therapeutic agents that target BCMA.

### BRIEF SUMMARY OF THE INVENTION

Methods of treating multiple myeloma comprising administering a gamma secretase inhibitor to a patient in need thereof wherein an amount of B-cell maturation antigen expressed on malignant plasma cell is increased by about 5% to about 70% are described herein.

Methods of treating multiple myeloma comprising administering a gamma secretase inhibitor to a patient in need thereof wherein an amount of B-cell maturation antigen expressed is about 90% on malignant plasma cells of the patient are also described herein.

Methods of treating multiple myeloma comprising administering a gamma secretase inhibitor to a patient in need thereof wherein an amount of B-cell maturation antigen expressed is more than 50% on malignant plasma cells of the patient are further described herein.

Methods of treating multiple myeloma comprising administering a gamma secretase inhibitor to a patient in need thereof wherein an amount of B-cell maturation antigen receptor density of the patient is increased by more than 5-fold are described herein.

Methods of treating multiple myeloma comprising administering a gamma secretase inhibitor to a patient in need thereof wherein an amount of soluble B-cell maturation antigen of the patient is reduced by more than 5% are also described herein.

In some aspects, the amount of B-cell maturation antigen expressed by the patient is determined from a blood sample of the patient. In some aspects, the amount of B-cell maturation antigen expressed by the patient is determined by flow cytometry.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) increasing the dosage of the BCMA-targeting therapy when the sBCMA concentration is greater in the second biological sample than the sBCMA concentration in the first biological sample are disclosed herein.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) administering a second BCMA-targeting therapy in addition to the first BCMA-targeting therapy when the sBCMA concentration is greater in the second biological sample than the sBCMA concentration in the first biological sample are also disclosed.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) administering a second BCMA-targeting therapy in addition to the first BCMA-targeting therapy when the sBCMA concentration is greater in the second biological sample than the sBCMA concentration in the first biological sample are additionally disclosed.

In some aspects, the step of administering a gamma secretase inhibitor comprises administering a 150 mg dose of the gamma secretase inhibitor. In some aspects, the step of administering a gamma secretase inhibitor comprises administering a 100 mg dose of the gamma secretase inhibitor.

In some aspects, the gamma-secretase inhibitor is selected from the group consisting of nirogacestat, crenigacestat, AL101, AL102, semagacestat, avagacestat, and ianabecestat. In some aspects, the gamma-secretase inhibitor is nirogacestat or pharmaceutically acceptable salt thereof.

In some aspects, the gamma-secretase inhibitor is nirogacestat or pharmaceutically acceptable salt thereof administered one, two, three, or four times per day.

Methods for treating multiple myeloma comprising once daily administration of 150 mg nirogacestat or a pharmaceutically acceptable salt thereof to a patient in need thereof are disclosed herein. Methods for treating multiple myeloma comprising once daily administration of 100 mg nirogacestat or a pharmaceutically acceptable salt thereof to a patient in need thereof are also disclosed herein. Methods for treating multiple myeloma comprising once daily administration of 50 mg nirogacestat or a pharmaceutically acceptable salt thereof to a patient in need thereof are additionally disclosed herein. Methods for treating multiple myeloma comprising administration to a patient in need thereof 200 mg per day of nirogacestat or a pharmaceutically acceptable salt thereof to a patient in need thereof are further disclosed herein. Methods for treating multiple myeloma comprising administration to a patient in need thereof 100 mg per day of nirogacestat or a pharmaceutically acceptable salt thereof to a patient in need thereof are disclosed herein.

Methods for treating multiple myeloma in a patient in need thereof comprising administering to the patient a 50 mg oral dosage of nirogacestat or a pharmaceutically acceptable salt thereof wherein the oral dosage provides a mean maximum drug plasma concentration (Cₘₐₓ) of more than 100 ng/ml are disclosed herein. Methods for treating multiple myeloma in a patient in need thereof comprising administering to the patient a 100 mg oral dosage of nirogacestat or a pharmaceutically acceptable salt thereof wherein the oral dosage provides a mean maximum drug plasma concentration (Cₘₐₓ) of more than 225 ng/ml are also disclosed herein. Methods for treating multiple myeloma in a patient in need thereof comprising administering to the patient a 50 mg oral dosage of nirogacestat or a pharmaceutically acceptable salt thereof wherein the oral dosage provides an in vivo area under the plasma curve (AUC_{inf}) of less than 700 ng h/ml are additionally disclosed herein. Methods for treating multiple myeloma in a patient in need thereof comprising administering to the patient a 100 mg oral dosage of nirogacestat or a pharmaceutically acceptable salt thereof wherein the oral dosage provides an in vivo area under the plasma curve (AUC_{inf}) of less than 3000 ng h/ml are further disclosed herein. Methods for treating multiple myeloma in a patient in need thereof comprising administering to the patient an oral dosage of nirogacestat or a pharmaceutically acceptable salt thereof wherein the oral dosage provides a mean maximum drug plasma concentration (Cₘₐₓ) of more than 100 ng/ml are disclosed herein. Methods for treating multiple myeloma in a patient in need thereof comprising administering to the patient an oral dosage of nirogacestat or a pharmaceutically acceptable salt thereof wherein the oral dosage provides a mean maximum drug plasma concentration (Cₘₐₓ) of more than 225 ng/ml are also disclosed herein. Methods for treating multiple myeloma in a patient in need thereof comprising administering to the patient an oral dosage of nirogacestat or a pharmaceutically acceptable salt thereof wherein the oral dosage provides an in vivo area under the plasma curve (AUC_{inf}) of less than 700 ng h/ml are disclosed herein. Methods for treating multiple myeloma in a patient in need thereof comprising administering to the patient an oral dosage of nirogacestat or a pharmaceutically acceptable salt thereof wherein the oral dosage provides an in vivo area under the plasma curve (AUC_{inf}) of less than 3000 ng h/ml are also disclosed herein.

In some aspects, methods for treating multiple myeloma in a patient in need thereof comprising administering to the patient an oral dosage of nirogacestat or a pharmaceutically acceptable salt thereof in combination with belantamab mafodotin are disclosed herein.

Methods for treating multiple myeloma in a patient in need thereof comprising administering of the patient an oral dosage of nirogacestat or a pharmaceutically acceptable salt thereof in combination with a B-cell maturation antigen (BCMA)-directed therapy to the patient.

In some aspects, the BCMA-directed therapy is one or more of an allogenic chimeric antigen receptor T cell therapy, an autologous chimeric antigen receptor T cell therapy, an immunotherapy, an antibody drug conjugate therapy, or a bispecific antibody therapy with dual specificity for BCMA and an immune-related target.

In some aspects, the nirogacestat, or pharmaceutically acceptable salt thereof, is nirogacestat hydrobromide. In some aspects, he nirogacestat hydrobromide is nirogacestat dihydrobomide.

In some aspects, the BCMA-directed therapy is one or more of an allogenic chimeric antigen receptor T cell therapy, an autologous chimeric antigen receptor T cell therapy, an immunotherapy, an antibody drug conjugate therapy, or a bispecific antibody therapy with dual specificity for BCMA and an immune-related target.

In some aspects, the gamma secretase is orally administered.

### BRIEF DESCRIPTION OF THE FIGURES

The accompanying drawings, which are incorporated herein and form a part of the specification, illustrate aspects of the present disclosure and, together with the description, further serve to explain the principles of the disclosure and to enable a person skilled in the pertinent art to make and use the disclosure.

FIG. 1 illustrates a diagram of a method for obtaining cell data measurements from a sample using a flow cytometer, according to aspects of the present disclosure.

FIG. 2 illustrates a flowchart diagram for a method of quantifying cells having expressed BCMA, according to aspects of the present disclosure.

FIG. 3 illustrates a flowchart diagram for a gating strategy to quantify cells having expressed BCMA, according to aspects of the present disclosure.

FIG. 4 illustrates a flowchart diagram for another gating strategy to quantify cells having expressed BCMA, according to aspects of the present disclosure.

FIG. 5 illustrates an example of gates that may be used in a gating strategy to isolate and quantify cells having expressed BCMA, according to aspects of the present disclosure.

FIG. 6 illustrates an example of gates that may be used in a gating strategy to isolate and quantify cells having expressed BCMA, according to aspects of the present disclosure.

FIG. 7 illustrates another example of gates that may be used in a gating strategy to isolate and quantify cells having expressed BCMA, according to aspects of the present disclosure.

FIG. 8 illustrates a block diagram of example components of a computer system, according to aspects of the present disclosure.

Aspects of the present disclosure will be described with reference to the accompanying drawings.

### DETAILED DESCRIPTION OF THE INVENTION

### I. Overview

B-cell maturation antigen (BCMA) is expressed on the surface of plasma cells and regulates their survival. In multiple myeloma, BCMA is widely expressed on malignant cells but is largely not expressed on normal tissues. BCMA can be released from the cell surface as soluble BCMA (sBCMA) which can be detected in the serum of patients with several different types of B cell malignancies. Levels of BCMA in the serum can be correlated with disease activity and overall survival of these patients.

Gamma secretase is involved in the cleavage of membrane bound BCMA and shedding of the BCMA's extracellular domain into the serum as soluble BCMA. BCMA shedding can create challenges for therapeutic agents that target BCMA. Some of the challenges include the following. First, BCMA shedding can decrease surface BCMA expression on cancer cells which then reduces target binding sites for BCMA-targeting therapeutic agents. Second, BCMA shedding can generate a soluble BCMA sink that binds to BCMA-targeting therapeutic agents and diverts these agents from binding to membrane bound BCMA expressed on cancer cells. Third, soluble BCMA molecules can also sequester circulating BCMA ligands, e.g., B-cell activating factor (BAFF) and a proliferation-inducing ligand (APRIL), and prevent them from stimulating BCMA expressed on the surface of B cells and plasma cells, thereby leading to deficient humoral immune responses in patients.

The use of gamma secretase inhibitors or pharmaceutical acceptably salts thereof to prevent BCMA shedding can increase the effectiveness of BCMA-directed therapies that target pathological B cells expressing BCMA. The present disclosure provides a method of treating multiple myeloma in a patient in need thereof comprising administering a gamma secretase inhibitor (e.g., nirogacestat) or pharmaceutically acceptable salt thereof and a BCMA-directed therapy to the patient.

### II. Definitions

To facilitate an understanding of the disclosure set forth herein, a number of terms and phrases are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “patient” refers to an animal, including, but not limited to, a primate (e.g., human), cow, sheep, goat, horse, dog, cat, rabbit, rat, or mouse.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. In certain aspects, a patient is successfully “treated” for multiple myeloma according to the methods of the present invention if the patient shows one or more of the following: a reduction in the number of or complete absence of cancer cells; relief of one or more symptoms associated with the specific cancer; reduced morbidity and mortality; improvement in quality of life; increased progression-free survival (PFS), disease-free survival (DFS), overall survival (OS), metastasis-free survival (MFS), complete response (CR), near complete response (nCR), stringent complete response (sCR), minor response (MR), minimal residual disease (MRD), partial response (PR), very good partial response (VGPR), stable disease (SD), a decrease in progressive disease (PD), an increased time to progression (TTP), or any combination thereof. In some aspects, the International Myeloma Working Group (IMWG) Uniform Response Criteria for Multiple Myeloma criteria can be used to determine whether the combination of a gamma secretase inhibit (e.g., nirogacestat) or a pharmaceutically acceptable salt thereof and the BCMA-directed therapy meets any of these particular endpoints (e.g., CR, nCR, sCR, MRD).

CR for a patient having multiple myeloma can be a negative immunofixation on the serum and urine and disappearance of any soft tissue plasmacytomas and <5% plasma cells in bone marrow.

sCR for a patient having multiple myeloma can be a CR plus normal serum free light chain (FLC) ratio and absence of clonal cells in bone marrow by immunohistochemistry or immunoflorence.

VGPR for a patient having multiple myeloma can be a serum and urine M-protein detectable by immunofixation but not on electrophoresis or >90% reduction in serum M-protein plus urine M-protein level<100 mg/24 h.

PD for a patient having multiple myeloma can be an increase of >25% from lowest response value in any one or more of the following:
serum M-component and/or (the absolute increase must be >0.5 g/dL);
urine M-component and/or (the absolute increase must be >200 mg/24 h);
only in patients without measurable serum and urine M-protein levels; the difference between involved and uninvolved FLC levels. The absolute increase must be >10 mg/dL;
bone marrow plasma cell percentage (the absolute percentage must be >10%);
definite development of new bone lesions or soft tissue plasmacytomas or definite increase in the size of existing bone lesions or soft tissue plasmacytomas;
development of hypercalcaemia (corrected serum calcium>11.5 mg/dL or 2.65 mmol/L) that can be attributed solely to the plasma cell proliferative disorder;

PR for a patient having multiple myeloma can be a >50% reduction of serum M-protein and reduction in 24 hours urinary M-protein by >90% or to <200 mg/24 h. If the serum and urine M-protein are unmeasurable, a >50% decrease in the difference between involved and uninvolved FLC levels can be required in place of the M-protein criteria. If the serum and urine M-protein are not measurable, and the serum free light assay can also not be measured, >50% reduction in plasma cells can be required in place of the M-protein, provided a baseline bone marrow plasma cell percentage was >30%. In addition, if present at baseline, a >50% reduction in the size of soft tissue plasmacytomas can also be required.

SD for a patient having multiple myeloma can be not meeting criteria for CR, VGPR, PR, or PD.

A patient having multiple myeloma that tests MRD negative has less than one myeloma cell per million bone marrow cells.

The terms “administering,” “administer,” or “administration” refer to delivering one or more compounds or compositions to a patient parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration.

The term “effective amount” refers to an amount of a compound, formulation, material, or composition, as described herein effective to achieve a particular biological result.

The term “therapeutically effective amount” includes the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

The terms “pharmaceutically acceptable carrier,” “pharmaceutically acceptable excipient,” “physiologically acceptable carrier,” or “physiologically acceptable excipient” refer to a pharmaceutically-acceptable material, composition, or vehicle, such as a liquid or solid filler, diluent, excipient, solvent, or encapsulating material. In one aspect, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The term “first line of therapy” as used throughout this disclosure, refers to a treatment regimen generally accepted or recommended by the medical establishment or a regulatory authority, e.g., the U.S. Food and Drug Administration or the European Medicines Agency, for the initial treatment of multiple myeloma in a patient. The patient having multiple myeloma can have previously received and/or be currently being treated for one or more unrelated diseases or disorders (e.g., anxiety).

It is understood that wherever embodiments are described herein with the language “comprising,” otherwise analogous embodiments described in terms of “consisting of” and/or “consisting essentially of” are also provided.

### III. Assay

BCMA is found on the surface of B-cells, which are produced in bone marrow. Sampling bone marrow is the most commonly practiced method to retrieve cells having expressed BCMA, because B-cells have a higher concentration in bone marrow than elsewhere in the body. There is also a well-known process of isolating and quantifying the cells having expressed BCMA in bone marrow using flow cytometry. With the interest in BCMA for discovery and treatment of certain diseases, however, it may be necessary to find alternative strategies for collecting BCMA because of the burden it places on the patients. The collection of bone marrow from a subject is expensive, invasive, and painful for the patient. Although it is known that flow cytometry can generally be completed on whole blood, there has been no innovation quantifying cells having expressed BCMA with a whole blood sample. There are challenges with this approach because B-cells are not as concentrated in whole blood, meaning there are lower concentrations of BCMA in the sample. This requires a new flow cytometry strategy for isolating cells having expressed BCMA to quantify their expression levels in a whole blood sample.

Isolating, quantifying, and/or measuring cells having expressed BCMA using whole blood samples can be useful in researching, studying, and testing the effects of disease therapies and/or treatment. Aspects described herein for quantifying cells having expressed BCMA can be used in many different applications of research and development, while also removing the need to collect bone marrow samples. For example, the method can be used in drug development, clinical studies and trials, testing for drug efficacy, and treatment plans for patients.

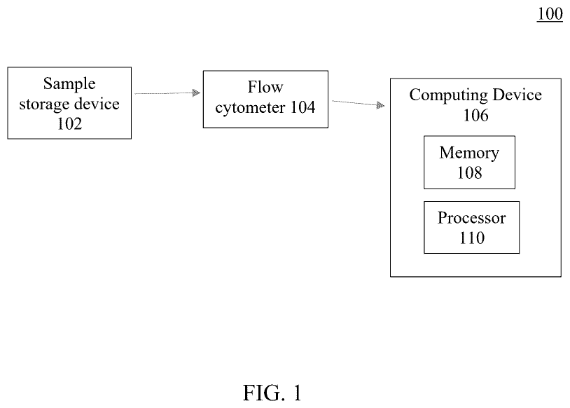

FIG. 1 illustrates a diagram of a system 100 for isolating and/or measuring, from whole blood, cells having a particular protein using a flow cytometer. In some aspects, system 100 is used to isolate, from whole blood, cells having surface BCMA, such that the BCMA levels on those cells can then be measured. System 100 includes one or more sample storage devices 102, a flow cytometer 104, and a computing device 106. Sample storage device 102 may be a physical storage device storing a biological sample, such as a sample tube, beaker, or pipette. The biological sample in the sample storage device may be, for example, a whole blood sample from a patient. In some aspects, the patient is a mammal, such as a human.

The biological sample in sample storage device 102 may be added to flow cytometer 104 for processing. Flow cytometer 104 uses light scattering and fluorescence on a liquid suspension containing cells to collect cell data on a cell-by-cell basis. The collected cell data may be used to classify each cell, count the cells, and/or make additional measurements pertaining to the cells.

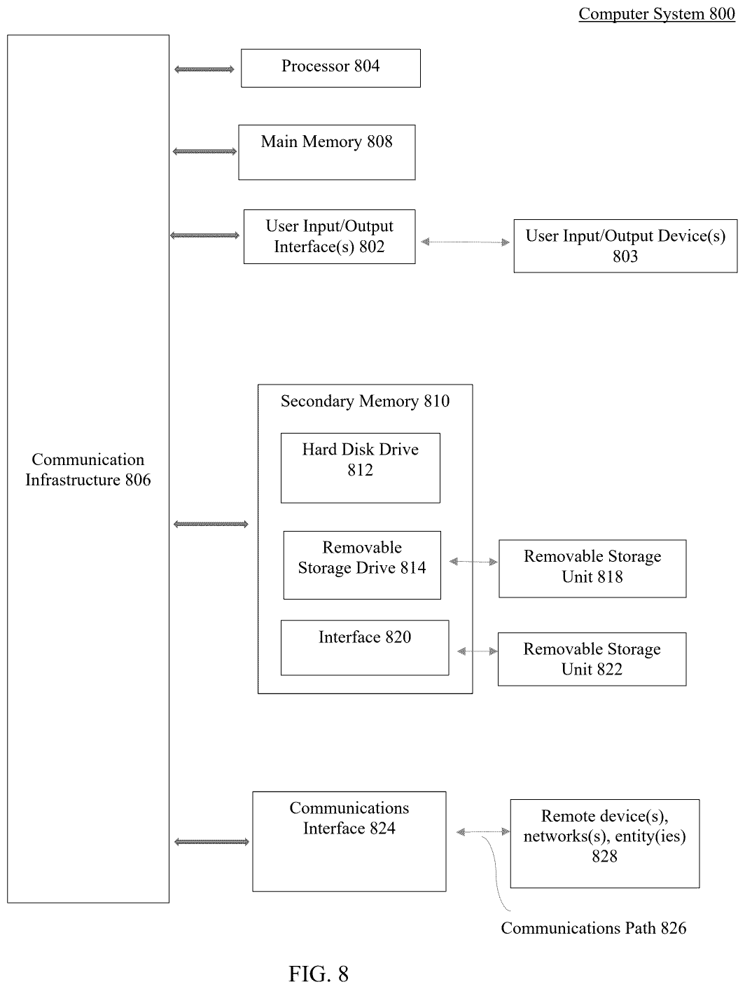

Computer 106 may include, among other things, a memory 108 and a processor 110. Further details regarding computer 106 are provided with reference to FIG. 8, below. Cell data from flow cytometer 104 may be stored in memory 108 of computing device 106. Processor 110 is configured to execute instructions stored in a computer-readable memory, such as memory 108, to process the cell data from flow cytometer 104.

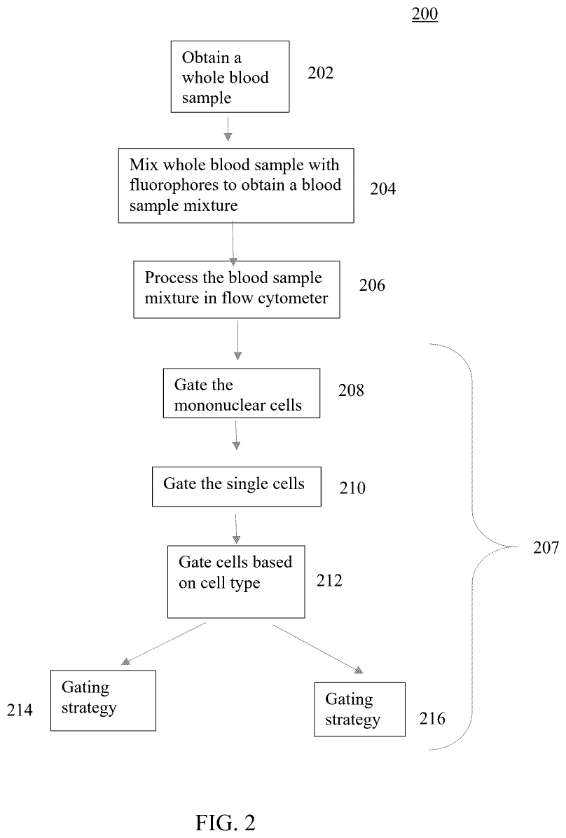

FIG. 2 illustrates a flowchart diagram of a method 200 for identifying, from whole blood, plasma cells having expressed BCMA. Method 200 begins with step 202, in which a whole blood sample is obtained. The whole blood sample may contain plasma cells having expressed BCMA, as well as other types of cells that would typically be found in whole blood. The whole blood sample may be obtained from a patient through any means of obtaining whole blood, such as a pin prick or blood draw. The whole blood sample may be contained in, for example, sample storage device 102.

To isolate cells using flow cytometry, fluorescently tagged molecules called fluorophores may be used to stain the surface of the cells in the whole blood sample. Fluorophores are microscopic molecules that absorb and emit fluorescent light. At step 204, the whole blood sample is mixed with a mixture of fluorophores that are configured to attach to a specific type of cell in the blood sample. In some aspects, the fluorophores may be used in conjunction with antibodies to create a fluorophore antibody reagent. Each antibody targets a specific type of cell or cell component, and the fluorophores attached to the antibody will stain the antibody's target cell. When targeting cells in a heterogeneous sample of cells, the fluorophore antibody reagents are mixed together into a fluorophore mixture to stain the different types of cells or cell components.

In some aspects, the fluorophore mixture that may be used to quantify cells having expressed BCMA includes fluorophore antibody reagents that detect, for example and without limitation, B-lymphocytes, monocytes, T-cells, natural killer cells, plasma cells, and BCMA. For example, the fluorophore mixture may include CD19 PerCP-Cy™5.5, CD14 FITC, CD3 FITC, CD56 FITC, CD138 APC, CD38 BV421, and CD269 PE. A person of skill in the art will recognize that the mixture may contain additional or fewer fluorophore reagents depending on the use case. A person of skill in the art will further recognize that a fluorophore reagent for targeting a specific type of cell may be swapped for another flurophore reagent that is also known to target that same specific type of cell.

The fluorophore mixture and the whole blood sample may be mixed such that each fluorophore antibody reagent in the fluorophore mixture will stain the antibody's targeted cell in the whole blood sample. This creates a whole blood sample mixture. The mixing may occur through any means of mixing two substances to obtain a whole blood sample mixture, such as a rotating mixer or a rolling mixer. The whole blood sample mixture may be held in sample storage device 102. In some aspects, the whole blood sample is mixed directly in sample storage device 102. In some other aspects, the whole blood sample is moved to an intermediate storage device (not shown) for mixing.

At step 206, the whole blood sample mixture is processed in the flow cytometer 104. Flow cytometry is a well-known process for acquiring specific information about individual cells, especially cells within a heterogeneous mixture. Flow cytometry can measure characteristics of cells in a sample, including size, count, and cell cycle. In some aspects, these measurements are taken by fluorescently labelling cell types or cell components in a sample and passing the cells in a single file through a laser. When the cell passes the laser, scattered light measurements and fluorescent light measurements are stored on a computing device in a computational dataset. The scattered light measurements can be used to measure, for example, size and granularity of a cell. The fluorescent light measurements are measurements of fluorescent labels on a cell that are excited and emit light at varying wavelengths when passed through the laser. In some aspects, a flow cytometer may include several detectors to measure different properties of the cell. A Forward Scatter (FSC) detector may be used to measure cell volume. A Side Scatter (SSC) detector may be used to measure granularity. Fluorescent detectors detect different cells or cell components based on the fluorescence they emit. In step 206, the whole blood sample mixture may be processed in the flow cytometer, and a computational dataset containing the whole blood sample's cell measurements may be collected and stored in a computing device for further analysis. For example, each cell in the whole blood sample mixture from sample storage device 102 may be measured by flow cytometer 104. The resulting computational dataset may be stored in memory 108 of computing device 106.

Once the scattered and fluorescent light measurements are collected and stored in the computational dataset in the computing device, they can be used to isolate and/or identify certain cells or cell components that are of interest. In some aspects of the present invention, a scatter plot may be used to compare two different light measurements of each cell in the computational dataset simultaneously. Certain areas of the scatter plot may signify a certain cell type. Selecting certain areas in the scatter plot to isolate a certain cell type is called gating. Gating sequentially selects areas on the scatter plot where the cells share similar measurements to determine which cells will be further analyzed and which cells will not be further analyzed. As referred to herein, a “positive” or “+” gate keeps those cells having the attribute being searched, while a “negative” or “−” gate keeps those cells that do not have the attribute being searched. When seeking to isolate specific or rare cells, there may be a series of gates, called a gating strategy, applied to the computational dataset. The sequence in which these gates are applied plays a key role in isolating cells of interest. If data corresponding to a particular set of cells is removed from the gating strategy, that data may either be deleted completely from the computational dataset, or flagged or recategorized such that it is simply not considered for future steps in the gating strategy.

BCMA, for example, is found on a rare cell type in whole blood. Accordingly, searching every cell in the biological sample for BCMA would be computationally intensive and time consuming. In accordance with aspects of the present invention, computational identification of these rare, BCMA-containing plasma cells can be made feasible by the gating strategies described herein because they classify/sort the cells in manageable stages through which relevant cells can be targeted. This reduces the computational complexity and thus reduces the time needed to efficiently identify BCMA-containing plasma cells. Accordingly, the gating strategies described herein are vital to isolating and quantifying the plasma cells having expressed BCMA, as there are lesser quantities of the cell in the whole blood sample.

The computational dataset generated by the flow cytometer may be processed through a gating strategy 207. In some aspects, computing device 106 may include tools for data acquisition and data analysis from flow cytometer 104, and may use processor 110 to process the computational dataset to identify cells having expressed BCMA in the whole blood sample mixture. In the aspect shown in FIG. 2, gating strategy 207 includes steps 208, 210, and 212, and one of step 214 and 216.

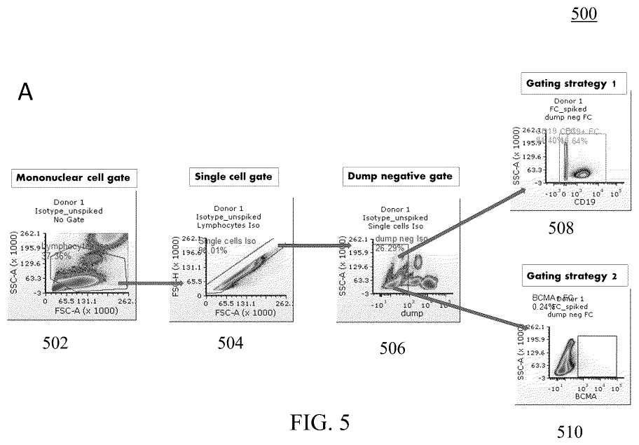

At step 208, the cells in the computational dataset may be gated by a mononuclear cell gate. A mononuclear cell gate included in the gating strategy may select cells that are mononuclear. Mononuclear cells refer to blood cells that have a single, round nucleus, including lymphocytes, which are the type of cells BCMA is found on. An example result from a mononuclear cell gate is illustrated in FIG. 5, plot 502. FIG. 5 is a graphical depiction of scatter plots from an example implementation of method 200 on a specific whole blood sample. As shown by plot 502, a mononuclear cell gate scatter plot may include FSC-area measurements and SSC-area measurements that measure size and granularity to select cells for further analysis. In this example, the mononuclear cell gate is a positive gate. The mononuclear cell gate may keep cells (that is, keep data corresponding to those cells) for further analysis that are within the range of size and shape of a mononuclear cell. Data for cells that do not present as mononuclear are removed from further analysis.

At step 210, the cells output from the mononuclear cell gate-the mononuclear cells-are gated by a single cell gate. A single cell gate included in the gating strategy may select cells that are single cells. A single cell gate is important because it removes cells that are stuck together, which may appear positive for antigens that would not be positive on a single cell, thus distorting the data. An example result from a single cell gate is illustrated in FIG. 5, plot 504. As shown by plot 504, a single cell gate scatter plot may include FSC-area measurements and FSC-height measurements that measure cell size to select cells for further analysis. In this example, the single cell gate is a positive gate. The single gate may keep cells that are within the range of size of a single cell. Data for cells that do not present as single cells are removed from further analysis.

A person of skill in the art will recognize that steps 208 and 210 may occur in any order. In some aspects, step 208 (mononuclear cell gate) occurs prior to step 210 (single cell gate), so that the cells remaining after the mononuclear cell gate are input into the single cell gate. In some other aspects, step 210 (single cell gate) occurs prior to step 208 (mononuclear cell gate), so that all cells are initially processed by the single cell gate, and the cells remaining after the single cell gate are input into the mononuclear cell gate. The cells remaining after passing through both the mononuclear gate and the single cell gate will be referred to herein as a first set of cells.

At step 212, cells that are not a certain cell type in the first set of cells are gated by a dump gate. A dump gate included in the gating strategy may select (keep) cells in the first set of cells that are not of a certain cell type. For example, the dump gate may select (keep) cells that are not monocytes, T-cells, or natural killer cells. Monocytes, T-cells, and natural killer cells are removed because BCMA is not found on those types of cells. The monocytes may be fluorescently marked (and thus identified by the dump gate) by, for example, CD14 fluorophores. The T-cells may be fluorescently marked by, for example, CD3 fluorophores. The natural killer cells may be fluorescently marked by, for example, CD56 fluorophores. Because each of these types of cells are marked, they can be removed from the data set by the dump gate. One of skill in the art will recognize that other fluorophores that identify monocytes, T-cells, and/or natural killer cells may alternatively or additionally be used. An example result from a dump gate is illustrated in FIG. 5, plot 506. As shown by plot 506, a dump gate scatter plot may include SSC-area measurements and fluorescent emissions of cells. In this example, the dump gate is a negative dump gate. The dump gate may keep all cells in the first set of cells that are not fluorescently marked by CD14 fluorophores, CD3 fluorophores, or CD56 fluorophores. The cells from the first set of cells that remain after the dump gate will be referred to herein as a second set of cells.

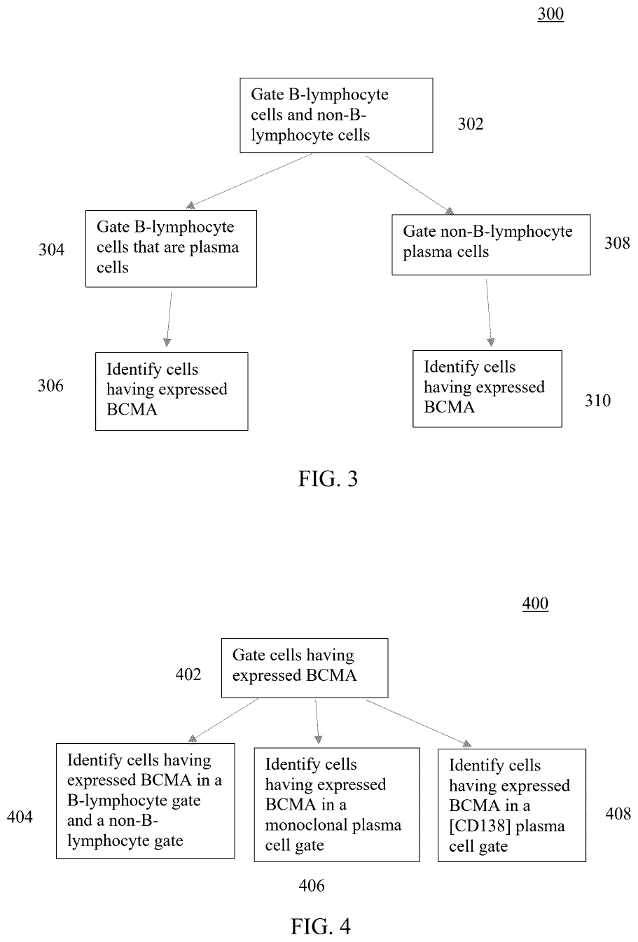

After step 212, the second set of cells may be processed through either gating strategy 214 or gating strategy 216 to further narrow down the cells to identify plasma cells having surface BCMA. Gating strategy 214 is described below with respect to FIG. 3, while gating strategy 216 is described below with respect to FIG. 4.

### Gating Strategy 1

FIG. 3 illustrates a flowchart diagram of a method 300 for the gating strategy 214. At step 302, the second set of cells are separated into two subsets of cells by a B-lymphocyte gate. One subset of cells includes B-lymphocytes and the other subset of cells includes non-B-lymphocytes. B-lymphocytes are selected because BCMA is found on B-lymphocytes. The B-lymphocytes may be fluorescently marked by, for example, CD19 fluorophores. One of skill in the art will recognize that a different fluorophore that identifies B-lymphocytes may alternatively or additionally be used.

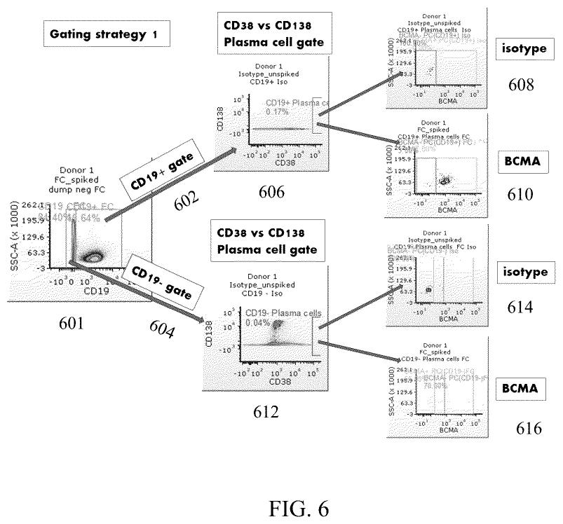

An example result from a B-lymphocyte gate is illustrated in FIG. 6, plot 601. As shown by plot 601, a scatter plot may include SSC-area measurements and CD19 fluorescence emissions. In this example, the B-lymphocyte gate is both a positive and a negative gate. Cells processed through the B-lymphocyte gate may be placed in a subset depending on whether the cell emits CD19 fluorescence (e.g., CD19+ gate 602) or does not emit CD19 fluorescence (e.g., CD19− gate 604). The B-lymphocyte gate may generate a B-lymphocyte cell subset and a non-B-lymphocyte cell subset.

Returning to FIG. 3, at step 304, cells in the B-lymphocyte cell subset are gated by a plasma cell gate. A plasma cell gate included in the gating strategy may select cells that are plasma cells. Plasma cells are selected because plasma cells develop from B-lymphocytes that have been activated, and BCMA is found on these activated B-lymphocytes. Plasma cells may be identified based on a co-expression of CD138 and CD38. Cells expressing CD38 may be fluorescently marked by CD38 fluorophores. Cells expressing CD138 may be fluorescently marked by CD138 fluorophores. Cells having the appropriate fluorescence emissions for CD138 fluorophores and CD38 fluorophores may be identified as plasma cells. The plasma cell gate may keep cells for further analysis that are identified as plasma cells. An example result from a plasma cell gate is illustrated in FIG. 6, plot 606. As shown by plot 606, a scatter plot may include fluorescence emissions for CD38 fluorophores and CD138 fluorophores so as to select an area of plasma cells. The plasma cell gate may be used to select cells that are plasma cells in the B-lymphocyte cell subset to generate a B-lymphocyte plasma cell subset.

At step 306, cells in the B-lymphocyte plasma cell subset are gated by a BCMA gate. A BCMA gate included in the gating strategy may identify cells left in the B-lymphocyte plasma cell subset that express BCMA, to isolate and/or quantify the cells having expressed BCMA. The cells having expressed BCMA may be fluorescently marked by, for example, CD269 fluorophores. An example result from a BCMA gate is illustrated in FIG. 6, plot 610. As shown by plot 610, a scatter plot may include SSC-area and fluorescence emissions for cells having expressed BCMA. The gate may be used to isolate and/or quantify the cells having expressed BCMA in the B-lymphocyte plasma cell subset.

Returning to FIG. 3, at step 308, cells in the non-B-lymphocyte cell subset are gated by a plasma cell gate. The plasma cell gate in step 308 may operate in a manner similar to that described for step 304. Cells expressing CD38 may be fluorescently marked by CD38 fluorophores. Cells expressing CD138 may be fluorescently marked by CD138 fluorophores. Cells having the appropriate fluorescence emissions for CD38 fluorophores and CD138 fluorophores may be identified as plasma cells in the non-B-lymphocyte cell subset to generate a non-B-lymphocyte plasma cell subset. An example result from a plasma cell gate acting on non-B-lymphocyte cells is illustrated in FIG. 6, plot 612. As shown by plot 612, a scatter plot may include fluorescence emissions for CD38 fluorophores and CD138 fluorophores so as to select an area of plasma cells.

At step 310, cells in the non-B-lymphocyte plasma cell subset are gated by a BCMA gate. The BCMA gate in step 310 may operate in a manner similar to that described for step 306. A BCMA gate included in the gating strategy may identify cells left in the non-B-lymphocyte plasma cell subset that express BCMA, to isolate and/or quantify the cells having expressed BCMA. An example result from a BCMA gate is illustrated in FIG. 6, plot 616. As shown by plot 616, the cell measurements analyzed in a scatter plot may include SSC-area and fluorescence emissions for cells having expressed BCMA. The cells having expressed BCMA may be fluorescently marked by, for example, CD269 fluorophores.

Once the cells having expressed BCMA (referred to herein as “BCMA cells”) are identified, various measurements can be made regarding the cells. For example, the quantity and/or percentage of the BCMA cells in the whole blood sample may be determined. In another example, the level of BCMA expression in the BCMA cells may be determined.

In some aspects, an isotype control may also be processed through the gating strategy to determine the accuracy of the gating strategy for quantifying cells having expressed BCMA. An isotype control may be mixed with the fluorophore mixture into an isotype sample mixture and processed in the flow cytometer to create an isotype computational dataset. The isotype computational dataset may be gated by the mononuclear gate, the single cell gate, the dump gate, the B-lymphocyte gate, the plasma cell gate, and the BCMA gate. The quantity of cells having expressed BCMA after applying the gating strategy may conclude that the gating strategy was accurately able to isolate cells having expressed BCMA in the whole blood sample mixture.

For example, in one aspect, an isotype sample mixture may be processed through the same steps of the gating strategy described above to determine the accuracy of the gating strategy for quantifying cells having expressed BCMA in the whole blood sample mixture. The isotype sample mixture may include a control isotype-PE and the fluorophore mixture. The isotype sample mixture may be processed by the flow cytometer 104 to produce an isotype computational dataset. The isotype computational dataset containing cell characterizations and measurements may be stored, for example, in memory 108 of computing device 106.

The isotype computational dataset may then be processed through the gating strategy of method 200 and method 300. Example results of processing the isotype computational dataset through the gating strategy of methods 200 and 300 are illustrated in FIG. 6, plots 608 and 614. Plot 608 illustrates a scatter plot from which the quantity of cells having expressed BCMA in a set of isotype B-lymphocyte plasma cells can be determined. Plot 614 illustrates a scatter plot from which the quantity of cells having expressed BCMA in a set of isotype non-B-lymphocyte plasma cells may be determined.

### Gating Strategy 2

As discussed above with respect to FIG. 2, after step 212, the second set of cells may be processed through either gating strategy 214 or gating strategy 216 to further narrow down the cells to identify plasma cells having surface BCMA.

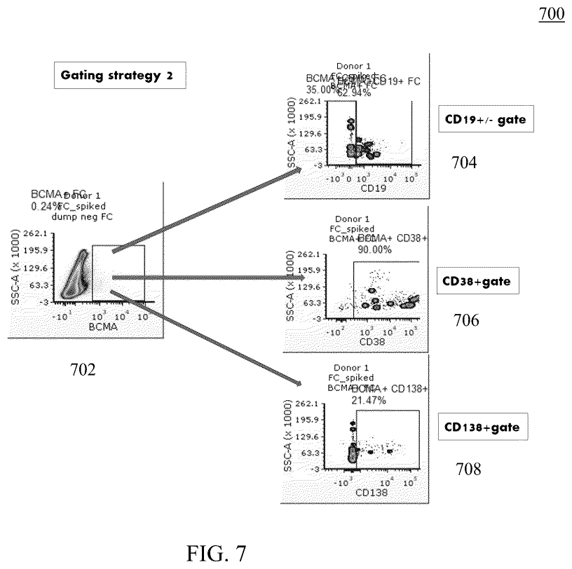

FIG. 4 illustrates a flowchart diagram of a method 400 for the gating strategy 216. At step 402, cells in the second set of cells are gated by a BCMA cell gate. A BCMA gate included in the gating strategy may select cells having expressed BCMA (referred to herein as “BCMA cells”) from the second set of cells. The cells having expressed BCMA may be fluorescently marked by, for example, CD269 fluorophores. An example result from a BCMA gate acting on the second set of cells is illustrated in FIG. 7, plot 702. As shown by plot 702, a BCMA gate scatter plot may include SSC-area measurements and fluorescence emissions for cells having expressed BCMA. In this example, the BCMA gate is a positive gate. The BCMA gate may be used to select (keep) cells that are fluorescently labelled as having expressed BCMA to generate a BCMA-positive cell subset. Data for cells in the second set of cells that do not present as expressing BCMA are removed from further analysis.

Cells in the BCMA-positive cell subset may be further processed by multiple additional gates to further classify the BCMA cells, as illustrated by steps 404, 406, and 408. Steps 404, 406, and 408 may be performed in parallel, or may be performed in any order.

At step 404, cells in the BCMA-positive cell subset are separated into two further subsets of cells by a B-lymphocyte gate. One subset of cells includes B-lymphocytes and the other subset of cells includes non-B-lymphocytes. The B-lymphocytes may be fluorescently marked by, for example, CD19 fluorophores. One of skill in the art will recognize that a different fluorophore that identifies B-lymphocytes may alternatively or additionally be used.

An example result from a B-lymphocyte gate is illustrated in FIG. 7, plot 704. As shown by plot 704, a scatter plot may include SSC-area measurements and CD19 fluorescence emissions. In this example, the B-lymphocyte gate is both a positive and a negative gate. BCMA cells processed through the B-lymphocyte gate may be placed in a subset depending on whether the cell emits CD19 fluorescence or does not emit CD19 fluorescence. The BCMA cells within each subset may then be identified, quantified, and/or measured.

At step 406, cells in the BCMA-positive cell subset are gated by a CD38 gate. Plasma cells that express CD38 may be fluorescently marked by CD38 fluorophores. An example result from a CD38 gate is illustrated in FIG. 7, plot 706. As shown by plot 706, a CD38 scatter plot may include SSC-area measurement and CD38 fluorescence emissions. Cells having expressed BCMA, as determined by their CD38 expression may be identified, quantified, and/or measured.

At step 408, cells in the BCMA-positive cell subset are gated by a CD138 gate. Plasma cells that express CD138 may be fluorescently marked by CD138 fluorophores. An example result from a CD138 gate is illustrated in FIG. 7, plot 708. As shown by plot 708, a CD138 scatter plot may include SSC-area measurement and CD138 fluorescence emissions. Cells having expressed BCMA, as determined by their CD138 expression may be identified, quantified, and/or measured.

### Example Computer System

FIG. 8 is a block diagram of example components of computer system 800. One or more computer systems 800 may be used, for example, to implement any of the aspects discussed herein, such as computer 106 discussed with reference to FIG. 1, as well as combinations and sub-combinations thereof. In some aspects, one or more computer systems 800 may be used to perform data acquisition, data analysis, and data processing, such as for the computational dataset obtained by flow cytometer 104 as described herein. Computer system 800 may include one or more processors (also called central processing units, or CPUs), such as a processor 804. Processor 804 may be connected to a communication infrastructure or bus 806.

Computer system 800 may also include user input/output interface(s) 802, such as monitors, keyboards, pointing devices, etc., which may communicate with communication infrastructure 806 through user input/output device(s) 803.

One or more of processors 804 may be a graphics processing unit (GPU). In an aspect, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 800 may also include a main or primary memory 808, such as random access memory (RAM). Main memory 808 may include one or more levels of cache. Main memory 808 may have stored therein control logic (i.e., computer software) and/or data.

Computer system 800 may also include one or more secondary storage devices or memory 810. Secondary memory 810 may include, for example, a hard disk drive 812 and/or a removable storage drive 814.

Removable storage drive 814 may interact with a removable storage unit 818. Removable storage unit 818 may include a computer usable or readable storage device having stored thereon computer software (control logic) and/or data. Removable storage unit 818 may be a program cartridge and cartridge interface (such as that found in video game devices), a removable memory chip (such as an EPROM or PROM) and associated socket, a memory stick and USB port, a memory card and associated memory card slot, and/or any other removable storage unit and associated interface. Removable storage drive 814 may read from and/or write to removable storage unit 818.

Secondary memory 810 may include other means, devices, components, instrumentalities or other approaches for allowing computer programs and/or other instructions and/or data to be accessed by computer system 800. Such means, devices, components, instrumentalities or other approaches may include, for example, a removable storage unit 822 and an interface 820. Examples of the removable storage unit 822 and the interface 820 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 800 may further include a communication or network interface 824. Communication interface 824 may enable computer system 800 to communicate and interact with any combination of external devices, external networks, external entities, etc. (individually and collectively referenced by reference number 828). For example, communication interface 824 may allow computer system 800 to communicate with external or remote devices 828 over communications path 826, which may be wired and/or wireless (or a combination thereof), and which may include any combination of LANs, WANs, the Internet, etc. Control logic and/or data may be transmitted to and from computer system 800 via communication path 826.

Computer system 800 may also be any of a personal digital assistant (PDA), desktop workstation, laptop or notebook computer, netbook, tablet, smartphone, smartwatch or other wearables, appliance, part of the Internet-of-Things, and/or embedded system, to name a few non-limiting examples, or any combination thereof.

In some aspects, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), may cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 8. In particular, aspects can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

Aspects of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### IV. Gamma Secretase Inhibitors

The present disclosure relates combination therapies comprising a gamma secretase inhibitor or a pharmaceutically acceptable salt thereof. In some aspects, the gamma secretase inhibitor is selected from the group consisting of nirogacestat, crenigacestat, AL101, AL102, semagacestat, avagacestat, and ianabecestat or a pharmaceutically acceptable salt thereof. In some aspects, the gamma secretase inhibitor is nirogacestat or a pharmaceutically acceptable salt thereof. In some aspects, the gamma secretase inhibitor is nirogacestat hydrobromide. In some aspects, the gamma secretase inhibitor is nirogacestat dihydrobromide.

The gamma secretase inhibitor (e.g., nirogacestat) or pharmaceutically acceptable salt thereof can be administered to patients via the oral, parenteral (such as subcutaneous, intravenous, intramuscular, intrasternal and infusion techniques), rectal, intranasal, topical or transdermal (e.g., through the use of a patch) routes. In one aspect, the gamma secretase inhibitor (e.g., nirogacestat) or pharmaceutically acceptable salt thereof can be administered to patients via the oral, parenteral (such as subcutaneous, intravenous, intramuscular, intrasternal and infusion techniques), rectal, intranasal, topical or transdermal (e.g., through the use of a patch) routes. In one aspect, the gamma secretase inhibitor (e.g., nirogacestat) or pharmaceutically acceptable salt thereof is orally administered. In one aspect, gamma secretase (e.g., nirogacestat) or pharmaceutically acceptable salt thereof is provided in tablet form.

In one aspect, the pharmaceutical composition comprises a gamma secretase (e.g., nirogacestat) or pharmaceutically acceptable salt thereof. In one aspect, the pharmaceutical composition is an oral tablet comprising a gamma secretase (e.g., nirogacestat) or pharmaceutically acceptable salt thereof and a pharmaceutically acceptable carrier. In one aspect, the tablet comprises about 10 mg to about 400 mg of the gamma secretase (e.g., nirogacestat) or a pharmaceutically acceptable salt thereof. In one aspect, the tablet comprises about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 225 mg, about 250 mg, about 275 mg, about 300 mg, about 325 mg, about 350 mg, about 375 mg, or about 400 mg of the gamma secretase inhibitor (e.g., nirogacestat) or a pharmaceutically acceptable salt thereof. In one aspect, the tablet comprises about 10 mg of the gamma secretase inhibitor (e.g., nirogacestat) or a pharmaceutically acceptable salt thereof. In one aspect, the tablet comprises about 20 mg of the gamma secretase inhibitor (e.g., nirogacestat) or a pharmaceutically acceptable salt thereof. In one aspect, the tablet comprises about 50 mg of the gamma secretase inhibitor (e.g., nirogacestat) or a pharmaceutically acceptable salt thereof. In one aspect, the tablet comprises about 100 mg of the gamma secretase inhibitor (e.g., nirogacestat) or a pharmaceutically acceptable salt thereof. In one aspect, the tablet comprises about 150 mg of the gamma secretase inhibitor (e.g., nirogacestat) or a pharmaceutically acceptable salt thereof. In one aspect, the tablet comprises about 200 mg of the gamma secretase inhibitor (e.g., nirogacestat) or a pharmaceutically acceptable salt thereof. In one aspect, the tablet comprises about 220 mg of the gamma secretase inhibitor (e.g., nirogacestat) or a pharmaceutically acceptable salt thereof.

For oral administration, known carriers can be included in the pharmaceutical composition. For example, microcrystalline cellulose, sodium citrate, calcium carbonate, dicalcium phosphate and glycine can be employed along with various disintegrants such as starch (preferably corn, potato or tapioca starch), methylcellulose, alginic acid and certain complex silicates, together with granulation binders such as polyvinylpyrrolidone, sucrose, gelatin and acacia, can be included in a tablet. Additionally, lubricating agents such as magnesium stearate, sodium lauryl sulfate and talc are often useful for tabletting purposes. Solid compositions of a similar type can also be employed as fillers in gelatin capsules. Preferred materials in this connection include lactose or milk sugar as well as high molecular weight polyethylene glycols. When aqueous suspensions and/or elixirs are desired for oral administration, the active ingredient can be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions containing nirogacestat can be prepared in either sesame or peanut oil, in aqueous propylene glycol, or in sterile water or saline. The aqueous solutions should be suitably buffered (preferably pH greater than 8) if necessary and the liquid diluent first rendered isotonic with sufficient saline or glucose. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

### V. B-Cell Maturation Antigen (BCMA)-Directed Therapy

In some aspects, the BCMA-directed therapy, includes but is not limited to, one or more of an allogeneic chimeric antigen receptor T cell therapy, an autologous chimeric antigen receptor T cell therapy, an immunotherapy (e.g., a monoclonal antibody therapy), an antibody drug conjugate therapy, or a bispecific antibody therapy with dual specificity for BCMA and an immune-related target (e.g., CD3). In some aspects, the BCMA-directed therapy can include at least an allogeneic chimeric antigen receptor T cell therapy. In some aspects, the BCMA-directed therapy can include at least an autologous chimeric antigen receptor T cell therapy. In some aspects, the BCMA-directed therapy can include at least an immunotherapy (e.g., a monoclonal antibody therapy). In some aspects, the BCMA-directed therapy can include at least an antibody drug conjugate. In some aspects, the BCMA-directed therapy can include at least a bispecific antibody therapy with dual specificity for BCMA and an immune-related target (CD3). In some aspects the BCMA-directed therapy includes any combination of the therapies listed above.

In some aspects, the BCMA-directed therapy can be formulated for intravenous or subcutaneous administration in a liquid dosage form.

### VI. Methods of Treatment

In one aspect, the combination of a gamma secretase inhibitor (e.g., nirogacestat) or a pharmaceutically acceptable salt thereof and BCMA-directed therapy is administered to treat multiple myeloma in a patient. In some aspects, methods of treating multiple myeloma comprising administering a gamma secretase inhibitor to a patient in need thereof wherein an amount of B-cell maturation antigen expressed on malignant plasma cell is increased by about 5% to about 70% are described herein.

In other aspects, methods of treating multiple myeloma comprising administering a gamma secretase inhibitor to a patient in need thereof wherein an amount of B-cell maturation antigen expressed is about 90% on malignant plasma cells of the patient are described herein.

In additional aspects, methods of treating multiple myeloma comprising administering a gamma secretase inhibitor to a patient in need thereof wherein an amount of B-cell maturation antigen expressed is more than 50% on malignant plasma cells of the patient are described herein.

In some aspects, methods of treating multiple myeloma comprising administering a gamma secretase inhibitor to a patient in need thereof wherein an amount of B-cell maturation antigen receptor density of the patient is increased by more than 5-fold are described herein.

Methods of treating multiple myeloma comprising administering a gamma secretase inhibitor to a patient in need thereof wherein an amount of soluble B-cell maturation antigen of the patient is reduced by more than 5% are also described herein.

In some aspects, the amount of B-cell maturation antigen expressed by the patient is determined from a blood sample of the patient. In some aspects, the amount of B-cell maturation antigen expressed by the patient is determined by flow cytometry.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) increasing the dosage of the BCMA-targeting therapy when the sBCMA concentration is greater in the second biological sample than the sBCMA concentration in the first biological sample are disclosed herein.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) administering a second BCMA-targeting therapy in addition to the first BCMA-targeting therapy when the sBCMA concentration is greater in the second biological sample than the sBCMA concentration in the first biological sample are also disclosed.

Methods of treating multiple myeloma in a patient in need thereof, comprising administering a combination therapy comprising a gamma secretase inhibitor and a B-cell maturation antigen (BCMA)-targeting therapy to the patient wherein the method comprises:
a) obtaining a first biological sample of whole blood from the patient;
b) determining a soluble B-cell maturation antigen (sBCMA) concentration in the first biological sample;
c) administering the gamma secretase inhibitor and the BCMA-targeting therapy to the patient;
d) obtaining a second biological sample of whole blood from the patient;
e) determining a sBCMA concentration in the second biological sample; and
f) administering a second BCMA-targeting therapy in addition to the first BCMA-targeting therapy when the sBCMA concentration is greater in the second biological sample than the sBCMA concentration in the first biological sample are additionally disclosed.

In some aspects, the patient with multiple myeloma exhibits a complete response following administration of the gamma secretase inhibitor (e.g., nirogacestat) or pharmaceutically acceptable salt thereof and BCMA-directed therapy. In some aspects, the patient with multiple myeloma exhibits a near complete response following administration of the gamma secretase inhibitor (e.g., nirogacestat) or pharmaceutically acceptable salt thereof and BCMA-directed therapy. In some aspects, the patient with multiple myeloma exhibits a stringent complete response following administration of the gamma secretase inhibitor (e.g., nirogacestat) or a pharmaceutically acceptable salt thereof and BCMA-directed therapy. In some aspects, the patient with multiple myeloma exhibits a minor response following administration of the gamma secretase inhibitor (e.g., nirogacestat) or a pharmaceutically acceptable salt thereof and BCMA-directed therapy. In some aspects, the patient with multiple myeloma exhibits a partial response following administration of the gamma secretase inhibitor (e.g., nirogacestat) or a pharmaceutically acceptable salt thereof and BCMA-directed therapy. In some aspects, the patient with multiple myeloma exhibits a very good partial response following administration of the gamma secretase inhibitor (e.g., nirogacestat) or a pharmaceutically acceptable salt thereof and BCMA-directed therapy. In some aspects, the patient with multiple myeloma exhibits stable disease following administration of the gamma secretase inhibitor (e.g., nirogacestat) or a pharmaceutically acceptable salt thereof and BCMA-directed therapy.

In one aspect, the gamma secretase inhibitor (e.g., nirogacestat) or a pharmaceutically acceptable salt thereof is administered to the patient with multiple myeloma before, concomitantly, or subsequently to the administering of the BCMA-directed therapy to the patient.

In one aspect, the patient with multiple myeloma is administered the combination therapy as the first line of therapy. In such aspect, the patient having multiple myeloma can have previously received and/or be currently being treated for one or more unrelated diseases or disorders (e.g., anxiety).

In some aspects, combination of gamma secretase inhibitor (e.g., nirogacestat) or a pharmaceutically acceptable salt thereof and the BCMA-directed therapy can be used in a combination with one or more of other known multiple myeloma treatments. In some aspects, the other known multiple myeloma treatments, include but are not limited to, a radiation therapy, a chemotherapy, a stem cell transplant an immunotherapy (e.g., a monoclonal antibody, such as a monoclonal antibody directed to CD38), a proteasome inhibitor, an immunomodulatory therapy, a hormone therapy, a photodynamic therapy, a targeted therapy (e.g., an XPO1 inhibitor), or a combination thereof. In some aspects, the other known cancer treatments can be an immunomodulatory therapy, a proteasome inhibitor, an immunotherapy (e.g., a monoclonal antibody, such as a monoclonal antibody directed to CD38), or a combination thereof. In some aspects, the other known cancer treatment can be a combination of an immunomodulatory therapy, a proteasome inhibitor, and an immunotherapy (e.g., a monoclonal antibody, such as a monoclonal antibody directed to CD38).

In one aspect, the patient has multiple myeloma after being previously treated for the multiple myeloma. In some aspects, the patient with multiple myeloma being treated with gamma secretase inhibitor (e.g., nirogacestat) or a pharmaceutically acceptable salt thereof and a BCMA-directed therapy has been previously treated for the multiple myeloma with one or more of a proteasome inhibitor, an immunomodulatory therapy, an immunotherapy (e.g., a monoclonal antibody, such as a monoclonal antibody directed to CD38), a stem cell transplant, a chemotherapy, a targeted therapy (e.g., an XPO1 inhibitor), a BCMA-directed therapy not in combination with a gamma secretase inhibitor (e.g., nirogacestat) or a pharmaceutically acceptable salt thereof to the patient, or combinations thereof. In one aspect, the patient has multiple myeloma after being previously treated for the multiple myeloma by a method comprising administration of a proteasome inhibitor to the subject. In one aspect, the patient has multiple myeloma after being previously treated for the multiple myeloma by a method comprising administration of an immunomodulatory therapy to the patient. In one aspect, the subject has multiple myeloma after being previously treated for the multiple myeloma by a method comprising administration of an immunotherapy (e.g., a monoclonal antibody, such as a monoclonal antibody directed to CD38) to the patient. In one aspect, the patient has multiple myeloma after being previously treated for the multiple myeloma by a method comprising a stem cell transplant to the patient. In one aspect, the patient has multiple myeloma after being previously treated for the multiple myeloma by a method comprising administration of a chemotherapy to the patient. In one aspect, the patient has multiple myeloma after being previously treated for the multiple myeloma by a method comprising administration of a targeted therapy (e.g., an XPO1 inhibitor) to the patient. In one aspect, the patient has multiple after being previously treated for the multiple myeloma by a method comprising administration of a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of a proteasome inhibitor and an immunomodulatory therapy to the patient. In one aspect, the patient has multiple myeloma after being previously treated for the multiple myeloma by a method comprising administration of the combination of a proteasome inhibitor and an immunotherapy (e.g., a monoclonal antibody, such as a monoclonal antibody directed to CD38) to the patient. In one aspect, the patient has multiple myeloma after being previously treated for the multiple myeloma by a method comprising a stem cell transplant and administration of a proteasome inhibitor to the patient. In one aspect, the patient has multiple myeloma after being previously treated for the multiple myeloma by a method comprising administration of the combination of a proteasome inhibitor and a chemotherapy to the patient. In one aspect, the subject has multiple myeloma or after being previously treated for the multiple myeloma by a method comprising administration of the combination of a proteasome inhibitor and a targeted therapy (e.g., an XPO1 inhibitor) to the patient. In one aspect, the patient has multiple myeloma after being previously treated for the multiple myeloma by a method comprising administration of the combination of a proteasome inhibitor and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of an immunomodulatory therapy and an immunotherapy (e.g., a monoclonal antibody, such as a monoclonal antibody directed to CD38) to the patient. In one aspect, the patient has multiple myeloma after being previously treated for the multiple myeloma by a method comprising a stem cell transplant and administration of an immunomodulatory therapy to the patient. In one aspect, the patient has multiple myeloma after being previously treated for the multiple myeloma by a method comprising administration of the combination of an immunomodulatory therapy and a chemotherapy to the patient. In one aspect, the patient has multiple myeloma after being previously treated for the multiple myeloma by a method comprising administration of the combination of an immunomodulatory therapy and a targeted therapy (e.g., an XPO1 inhibitor) to the patient. In one aspect, the patient has multiple myeloma after being previously treated for the multiple myeloma by a method comprising administration of the combination of an immunomodulatory therapy and a BCMA-directed therapy not in combination with a gamma secretase inhibitor (e.g., nirogacestat) or a pharmaceutically acceptable salt thereof to the patient. In one aspect, the subject has multiple myeloma after being previously treated for the multiple myeloma by a method comprising a stem cell transplant and administration of an immunotherapy (e.g., a monoclonal antibody, such as a monoclonal antibody directed to CD38) to the patient. In one aspect, the patient has multiple myeloma after being previously treated for the multiple myeloma by a method comprising administration of the combination of an immunotherapy (e.g., a monoclonal antibody, such as a monoclonal antibody directed to CD38) and a chemotherapy to the patient. In one aspect, the patient has multiple myeloma after being previously treated for the multiple myeloma by a method comprising administration of the combination of an immunotherapy (e.g., a monoclonal antibody, such as a monoclonal antibody directed to CD38) and a targeted therapy (e.g., an XPO1 inhibitor) to the patient. In one aspect, the patient has multiple myeloma after being previously treated for the multiple myeloma by a method comprising administration of the combination of an immunotherapy (e.g., a monoclonal antibody, such as a monoclonal antibody directed to CD38) and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma amyloidosis by a method comprising a stem cell transplant and administration of a chemotherapy to the patient. In one aspect, the patient has multiple myeloma after being previously treated for the multiple myeloma by a method comprising a stem cell transplant and administration of a targeted therapy (e.g., an XPO1 inhibitor) to the patient. In one aspect, the patient has multiple myeloma after being previously treated for the multiple myeloma by a method comprising a stem cell transplant and administration of a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of a chemotherapy and a targeted therapy (e.g., an XPO1 inhibitor) to the patient. In one aspect, the patient has multiple myeloma after being previously treated for the multiple myeloma by a method comprising administration of the combination of a chemotherapy and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of a targeted therapy (e.g., an XPO1 inhibitor) and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of a proteasome inhibitor, an immunomodulatory therapy, and an immunotherapy (e.g., a monoclonal antibody, such as a monoclonal antibody directed to CD38) to the patient. In one aspect, the patient has multiple myeloma after being previously treated for the multiple myeloma by a method comprising a stem cell transplant and administration of the combination of a proteasome inhibitor and an immunomodulatory therapy to the patient. In one aspect, the patient has multiple myeloma after being previously treated for the multiple myeloma by a method comprising administration of the combination of a proteasome inhibitor, an immunomodulatory therapy, and a chemotherapy to the patient. In one aspect, the patient has multiple myeloma after being previously treated for the multiple myeloma by a method comprising administration of the combination of a proteasome inhibitor, an immunomodulatory therapy, and a targeted therapy (e.g., an XPO1 inhibitor) to the patient. In one aspect, the patient has multiple myeloma after being previously treated for the multiple myeloma by a method comprising administration of the combination of a proteasome inhibitor, an immunomodulatory therapy, and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising a stem cell transplant and administration of the combination of a proteasome inhibitor and an immunotherapy (e.g., a monoclonal antibody, such as a monoclonal antibody directed to CD38). In one aspect, the patient has multiple myeloma after being previously treated for the multiple myeloma by a method comprising administration of the combination of a proteasome inhibitor, an immunotherapy (e.g., a monoclonal antibody, such as a monoclonal antibody directed to CD38), and a chemotherapy to the patient. In one aspect, the patient has multiple myeloma after being previously treated for the multiple myeloma by a method comprising administration of the combination of a proteasome inhibitor, an immunotherapy (e.g., a monoclonal antibody, such as a monoclonal antibody directed to CD38), and a targeted therapy (e.g., an XPO1 inhibitor) to the patient. In one aspect, the patient has multiple myeloma after being previously treated for the multiple myeloma by a method comprising administration of the combination of a proteasome inhibitor, an immunotherapy (e.g., a monoclonal antibody, such as a monoclonal antibody directed to CD38), and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising a stem cell transplant and administration of the combination of an immunomodulatory therapy and an immunotherapy (e.g., a monoclonal antibody, such as a monoclonal antibody directed to CD38) to the patient. In one aspect, the patient has multiple myeloma after being previously treated for the multiple myeloma by a method comprising administration of the combination of an immunomodulatory therapy, an immunotherapy (e.g., a monoclonal antibody, such as a monoclonal antibody directed to CD38), and a chemotherapy to the patient. In one aspect, the patient has multiple myeloma after being previously treated for the multiple myeloma by a method comprising administration of the combination of an immunomodulatory therapy, an immunotherapy (e.g., a monoclonal antibody, such as a monoclonal antibody directed to CD38), and a targeted therapy (e.g., an XPO1 inhibitor) to the patient. In one aspect, the patient has multiple myeloma after being previously treated for the multiple myeloma by a method comprising administration of the combination of an immunomodulatory therapy, an immunotherapy (e.g., a monoclonal antibody, such as a monoclonal antibody directed to CD38), and a BCMA-directed therapy not in combination with a gamma secetase inhibitor (e.g., nirogacestat) to the patient. In one aspect, the patient has multiple myeloma after being previously treated for the multiple myeloma by a method comprising a stem cell transplant and administration of the combination of an immunotherapy (e.g., a monoclonal antibody, such as a monoclonal antibody directed to CD38) and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of an immunotherapy (e.g., a monoclonal antibody, such as a monoclonal antibody directed to CD38), a chemotherapy, and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of an immunotherapy (e.g., a monoclonal antibody, such as a monoclonal antibody directed to CD38), a targeted therapy (e.g., an XPO1 inhibitor), and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising a stem cell transplant and administration of the combination of a chemotherapy and a targeted therapy (e.g., an XPO1 inhibitor) to the patient. In one aspect, the patient has multiple myeloma after being previously treated for the multiple myeloma by a method comprising a stem cell transplant and administration of the combination of a chemotherapy and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma amyloidosis by a method comprising a stem cell transplant and administration of the combination of a targeted therapy (e.g., an XPO1 inhibitor) and a BCMA-directed therapy not in combination with a gamma secretase inhibitor (e.g., nirogacestat) or a pharmaceutically acceptable salt thereof to the patient. In one aspect, the subject has multiple myeloma after being previously treated for the multiple myeloma by a method comprising administration of the combination of a chemotherapy, a targeted therapy (e.g., an XPO1 inhibitor), and a BCMA-directed therapy not in combination with a gamma secretase inhibitor (e.g., nirogacestat) or a pharmaceutically acceptable salt thereof to the patient. In one aspect, the patient has multiple myeloma after being previously treated for the multiple myeloma by a method comprising administration of the combination of a proteasome inhibitor, an immunomodulatory therapy, an immunotherapy (e.g., a monoclonal antibody such as a monoclonal antibody directed to CD38), and a BCMA-directed therapy not in combination with a gamma secretase inhibitor (e.g., nirogacestat) or a pharmaceutically acceptable salt thereof to the patient.

In one aspect, the methods of treating multiple myeloma comprise administering a gamma secretase inhibitor to a patient in need thereof wherein an amount of B-cell maturation antigen expressed on malignant plasma cell is increased by about 5% to about 70%. In some aspects, the amount of B-cell maturation antigen expressed on malignant plasma cell is increased by about 10% to about 65%, about 15% to 60%, about 20% to about 55%. In some aspects, the amount of B-cell maturation antigen expressed on malignant plasma cell is increased about 5%, about 10%, about 15%, about 20%, about 25%, about 30%, about 35%, about 40%, about 45%, about 55%, about 60%, about 65%, or about 70%.

In some aspects, the methods of treating multiple myeloma comprise administering a gamma secretase inhibitor (e.g., nirogacestat) or a pharmaceutically acceptable salt thereof to a patient in need thereof wherein an amount of B-cell maturation antigen expressed more than 50% on malignant plasma cells of the patient. In some aspects, the amount of B-cell maturation antigen expressed is more than 55%, 60%, 65%, 70%, or 75% on malignant plasma cells of the patient.

In other aspects, the methods of treating multiple myeloma comprise administering a gamma secretase inhibitor (e.g., nirogacestat) or a pharmaceutically acceptable salt thereof to a patient in need thereof wherein an amount of B-cell maturation antigen receptor density of the patient is increased by more than 5-fold. In some aspects, the amount of B-cell maturation antigen receptor density of the patient is increased by more than 6-fold, 7-fold, 8-fold, 9-fold, or 10-fold.

In some aspects, the methods of treating multiple myeloma comprise administering a gamma secretase inhibitor (e.g., nirogacestat) or a pharmaceutically acceptable salt thereof to a patient in need thereof wherein an amount of soluble B-cell maturation antigen of the patient is reduced by more than 5%. In additional aspects, the amount of soluble B-cell maturation antigen of the patient is reduced by more than 10%, 15%, 20%, or 25%.

In one aspect, the gamma secretase inhibitor (e.g., nirogacestat) or a pharmaceutically acceptable salt thereof or pharmaceutically acceptable salt thereof is administered in doses ranging from about 0.1 mg to about 1000 mg daily. In one aspect, the patient is administered about 50 mg to about 500 mg of gamma secretase inhibitor (e.g., nirogacestat) or a pharmaceutically acceptable salt thereof or pharmaceutically acceptable salt thereof daily. In another aspect, the patient is administered about 100 mg to about 400 mg of gamma secrease inhibitor (e.g., nirogacestat) or pharmaceutically acceptable salt thereof daily. In another aspect, the patient is administered about 20 mg to about 220 mg of nirogacestat dihydrobromide daily. In another aspect, the patient is administered about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 220 mg, about 225 mg, about 250 mg, about 275 mg, about 300 mg, about 325 mg, about 350 mg, about 375 mg, or about 400 mg daily of gamma secretase inhibitor (e.g., nirogacestat) or pharmaceutically acceptable salt thereof.

In one aspect, the gamma secretase inhibitor or pharmaceutically acceptable salt thereof is nirogacestat. In some aspects, the nirogacestat is nirogacestat hydrobromide. In some aspects, the nirogacestat hydrobromide is nirogacestat dihydrobromide. nirogacestat dihydrobromide is administered in doses ranging from about 0.1 mg to about 1000 mg daily. In one aspect, the patient is administered about 50 mg to about 500 mg of nirogacestat dihydrobromide daily. In another aspect, the subject is administered about 100 mg to about 400 mg of nirogacestat dihydrobromide daily. In another aspect, the subject is administered about 20 mg to about 220 mg of nirogacestat dihydrobromide daily. In another aspect, the subject is administered about 20 mg, about 25 mg, about 30 mg, about 40 mg, about 50 mg, about 60 mg, about 70 mg, about 75 mg, about 100 mg, about 125 mg, about 150 mg, about 175 mg, about 200 mg, about 220 mg, about 225 mg, about 250 mg, about 275 mg, about 300 mg, about 325 mg, about 350 mg, about 375 mg, or about 400 mg daily of nirogacestat dihydrobromide.

The total daily dose can be provided as single or divided doses (i.e., 1, 2, 3, or 4 doses per day). In one aspect, the total daily dose is provided as two doses. For example, a 300 mg or 200 mg total daily dose can be administered to a the as two separate 150 mg or 100 mg doses, respectively. In one aspect, three tablets comprising 50 mg of nirogacestat dihydrobromide twice daily or 200 mg daily dose can be administered to a subject as two tablets comprising 50 mg of gamma secretase inhibitor (e.g., nirogacestat) or pharmaceutically acceptable salt thereof twice daily.

In one aspect, the gamma secretase inhibitor (e.g., nirogacestat), or pharmaceutically acceptable salt thereof is administered orally and the BCMA-directed therapy is administered intravenously or subcutaneously to the subject.

In one aspect, the patient is human.

### EXAMPLES

### Example 1: Assessment of BCMA Expression in Human Multiple Myeloma Cell Lines Following Treatment with Nirogacestat Dihydrobromide

The BCMA-expressing multiple myeloma cells lines MM.1S, Molp-8, H929, and OPM2, and the BCMA-negative acute lymphocytic leukemia cell line REH, will be expanded in RPMI medium containing L-glutamine and 5 to 10% FBS in a humidified CO₂ incubator set to 37° C. Cells will be transferred to 96-well plates (1×10⁶ cells/mL) and cultured in the presence of increasing concentrations (0.01 nM to 3000 nM) of nirogacestat dihydrobromide or vehicle (control) in a humidified CO₂ incubator set to 37° C. for 5 to 24 hours. Cells will be harvested by centrifugation for 5 minutes at 400×g and washed with an appropriate buffer. Cells will then be suspended in 100 μL an appropriate buffer containing an anti-human BCMA antibody and stained for 30 to 60 minutes at 4° C. Cells will be washed twice with an appropriate buffer for flow cytometry analysis. The cell viability will be determined by a commercial assay as described by the manufacturer. Expression levels of BCMA (mean fluorescence intensity) will be determined by flow cytometry.

### Example 2: Assessment of sBCMA Shedding in Multiple Myeloma Cell Lines Following Treatment with Nirogacestat Dihydrobromide

The BCMA-expressing multiple myeloma cells lines MM.1S, Molp-8, H929, and OPM2, and the BCMA-negative acute lymphocytic leukemia cell line REH, will be expanded in RPMI medium containing L-glutamine and 5 to 10% FBS in a humidified CO₂ incubator set to 37° C. Cells will be transferred to 96-well plates (1×10⁶ cells/mL) and cultured in the presence of increasing concentrations (0.01 nM to 3000 nM) of nirogacestat dihydrobromide or vehicle (control) in a humidified CO₂ incubator set to 37° C. for 5 to 24 hours. Cell culture media will be collected throughout and/or following a specified time and analyzed for concentration of sBCMA using a commercially available sBCMA ELISA kit according to the instructions provided by the manufacturer.

### Example 3

The BCMA-expressing multiple myeloma cells lines MM.1S, Molp-8, H929, and OPM2, and the BCMA-negative acute lymphocytic leukemia cell line REH, will be expanded in RPMI medium containing L-glutamine and 5 to 10% FBS in a humidified CO₂ incubator set to 37° C. Cells will be transferred to 96-well plates (1×10⁶ cells/mL) and cultured in the presence of a fixed dose (e.g., 1 μM) of nirogacestat dihydrobromide or vehicle (control) in a humidified CO₂ incubator set to 37° C. Targeted BCMA therapies may be added a range of concentrations to evaluate the effects of the combination on the proliferation of the multiple myeloma cells in a 3-day cellular proliferation assay (e.g. Cell-Titre Glo).

### Example 4

Antibody-dependent cellular cytotoxicity (ADCC) activity of BCMA targeted antibodies will be determined using a BCMA directed IgGI monoclonal antibody in combination with nirogacestat dihydrobromide. ADCC activity against BCMA-expressing multiple myeloma cells lines (e.g., MM.1S, Molp-8, RPMI8226, ARH77, GA10, LP1, L363) will be measured using commercially available assays (e.g., Promega Jurkat ADCC assay) where a range of concentrations of nirogacestat dihydrobromide are combined with a range of concentrations of the BCMA targeted monoclonal antibody.

### Example 5

Bispecific cytotoxicity assays will be performed by mixing purified human CD3+ T cells and luciferase-labeled myeloma cell lines, E:T of 5:1, and serial dilutions of bispecific antibody. After 2 days of incubation, viability of cells will be assessed by OneGlo luciferase reagent (Promega).

### Example 6

T-cell dependent cellular cytotoxicity (TDCC) activity of BCMA×CD3 bispecific antibody will be determined in combination with nirogacestat dihydrobromide. Assays will be performed by mixing CD3+ T cells and luciferase-labeled multiple myeloma cell lines (e.g., MM.1S, Molp-8, RPMI8226, ARH77, GA10, LP1, L363) using an effector-to-target ratio of 5 to 1. Serial dilutions of the bispecific antibody and nirogacestat dihydrobromide will result in a range of concentrations of each molecule being evaluated. After 2 days of incubation, viability of cells will be assessed using a luciferase-based assay (Promega OneGlo).

### Example 7

T-cell dependent cellular cytotoxicity (TDCC) activity of BCMA targeted chimeric antigen T-cell (CAR-T) cells will be determined in combination with Form A of nirogacestat dihydrobromide. TDCC activity against BCMA-expressing multiple myeloma cells lines (e.g., MM.1S, Molp-8, RPMI8226, ARH77, GA10, LP1, L363) will be measured using custom developed TDCC assays (similar to the format described by Nazarian, A.A., et al., J. Biomol. Screen, 20:519-27 (2015)) where a range of concentrations of nirogacestat dihydrobromide will be combined with a range of BCMA targeted CAR-T cell numbers.

### Example 8

T-cell activation by BCMA targeted therapies (CAR-T cells, bispecific antibodies and monoclonal antibodies) in the presence of BCMA expressing multiple myeloma cell lines (e.g., MM.1S, Molp-8, RPMI8226, ARH77, GA10, LP1, L363) will be determined in combination with nirogacestat dihydrobromide. Co-cultures of T-cells and multiple myeloma cell lines will be incubated with fixed concentrations of nirogacestat dihydrobromide. Serial dilutions of BCMA targeted therapies will be added and T-cell activation will be determined by cytokine release assays and/or flow cytometry.

### Example 9: A Three-Part, Open Label, Randomized, Parallel Assignment Study to Evaluate the Pharmacodynamics of Nirogacestat on B-Cell Maturation Antigen (BCMA)

The study will be conducted in male participants to evaluate the safety, tolerability, PD, and PK profile of nirogacestat on BCMA. The primary endpoints include:
Evaluate the pharmacodynamics (PD) of nirogacestat dihydrobromide on BCMA. Additionally, the serum nirogacestat exposure-response relation to membrane-bound BCMA (mbBCMA) and soluble BCMA (sBCMA) kinetis after a single dose of nirogacestat dihydrobromide is administered.
The PD endpoints include (1) measurement of plasma cells in bone marrow (BM) and whole blood (WD) samples, (2) relative percentage of mbBCMA+Plasma Cells in BM and WB samples, (3) relative percentage of mbBCMA-Plasa Cells in BM and WB, (4) mbBCMA antibody binding densiy on Plasma Cells from BM and WB, and (5) serume soluble BCMA (sBCMA) levels.
The PKPD model describing the relationships between nirogacestat exposure and mbBCMA on plasma cells and sBCMA in serum will be determined.

Secondary endpoints will be:
Evaluate the pharmacokinetics of serum nirogacestat afer single and multiple dose administration of nirogacestat
Evaluate the safety and tolerability of single and multiple dose nirogacestat in healthy male participants
PK parameters
   AUCₜ (where t is the time of BM collection)
   AUCₗₐₛₜ
   Cₘₐₓ
   Tₘₐₓ
Safety assessments will include reporting of adverse events (AEs), clinical laboratory tests, 12-lead electrocardiograms (ECGs) vital signs, and physical examinations

Exploratory endpoints will be:
Establish an assay to measure mbBCMA antibody binding density on Plasma Cells from WB in healthy participatns
Evaluate gene expression in plasma cells that correlate gene expression in plasma cells that correlate with response to nirogacestat administration
Assay qualification as determined by perspecified acceptance criteria
Quantiatively measure RNA expression

Overall design: This will be a three-part, open-label, randomized, parallel design, Phase 1 study to evaluate the pharmacodynamics (PD), pharmacokinetics (PK), safety and tolerability of nirogacestat on B-cell maturation antigen (BCMA) in healthy adult men.

Part 1 of the study will be conducted to obtain a fresh bone marrow (BM) sample and a fresh whole blood (WB) sample for qualification of a flow cytometry assay that will be used in subsequent study parts to measure membrane-bound BCMA (mbBCMA) on plasma cells. Part 1 will include a screening phase of up to 35 days prior to enrollment on Day 1. Two eligible participants will be admitted to the clinical research unit (CRU) on Day −1. No drug will be administered, and a single BM aspirate and matching WB sample will be collected on Day 1. The participants will be discharged after a safety observation period of up to 1 hour following the BM aspirate. Participants will be instructed to follow-up with the CRU if any AEs occur within 30 days of the BM aspirate. The BM and WB samples will be submitted to a flow cytometry lab for assay qualification and evaluation. If it is determined that additional BM and WB samples are required for assay qualification, an additional participant may be enrolled in Part 1 to obtain sufficient material to continue the assay qualification. Upon qualification of the assay, Part 2 and Part 3 of the study may be initiated.

Part 2 and Part 3 of the study will include a screening phase of up to 35 days prior to dosing on Day 1. Eligible participants will be admitted to the CRU on Day −2 and will be discharged after a safety observation period of at least 1 hour following the participant's last BM aspirate/WB sample and upon completion of final safety assessments, (Day 2 or Day 3). A follow-up (FU) telephone call will be performed 30 to 32 days after the last dose of study treatment.

During Screening for Part 2 of the study, participants will sign the ICF prior to any study procedures being performed. Participants must satisfy all the inclusion and exclusion criteria to be eligible for study participation. Participants will be admitted to the CRU on Day −2 for check-in procedures and eligibility confirmation. On Day −1, 8 participants will be randomized to an assessment sequence (2:2:2:2) and the baseline BM aspirate/WB sample will be collected. Following an overnight fast of at least 8 hours, a single dose of 150 mg nirogacestat (50 mg×3 tablets) will be orally administered on Day 1. Breakfast may be administered to participants after the 2-hour PK collection. Participants will remain domiciled at the CRU until the participant's last BM aspirate, WB, and PK samples are collected and safety evaluations are completed (Day 2 or Day 3). A FU telephone call will be performed 30 to 32 days after the last dose of study treatment. Additional safety evaluations may be scheduled at the discretion of the Investigator prior to the FU telephone visit.

Upon completion of the treatment assessments for Part 2 of the study, an interim analysis will be conducted. The PKPD model describing the relationship between nirogacestat exposure and mbBCMA will be re-evaluated based on the data collected in Part 2. An adaptive enrollment approach will be taken for the initiation of Part 3 based on observed changes in mbBCMA and predictions utilizing the re-evaluated model. If predicted changes to mbBCMA at some timepoints are not found to be meaningful or helpful in determining a PKPD relationship, then some of the timepoints may be altered or eliminated. Simulations from the PKPD model will also be used to select additional dose regimens and timepoints for evaluation in Part 3 that may be tested to further qualify the PKPD model. Up to 10 participants will be enrolled into Part 3 and randomized to one post-dose BM timepoint at one of several possible doses or dose regimens which may be conducted. Enrollment in Part 3, as well as further collection of either BM or WB samples, may also be discontinued based on the results of the interim analysis.

It is to be appreciated that the Detailed Description section, and not the Summary and Abstract sections, is intended to be used to interpret the claims. The Summary and Abstract sections set forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method of treating multiple myeloma comprising administering a gamma secretase inhibitor to a patient in need thereof wherein (a) an amount of B-cell maturation antigen expressed on malignant plasma cell is increased by about 5% to about 70%, (b) an amount of B-cell maturation antigen expressed is about 90% on malignant plasma cells of the patient. (c) an amount of B-cell maturation antigen expressed is more than 50% on malignant plasma cells of the patient, (d) an amount of B-cell maturation antigen receptor density of the patient is increased by more than 5-fold, or (e) an amount of soluble B-cell maturation antigen of the patient is reduced by more than 5%.

2-5. (canceled)

6. The method of claim 1, wherein the amount of B-cell maturation antigen expressed by the patient is determined from a blood sample of the patient.

7. The method of claim 1, wherein the amount of B-cell maturation antigen expressed by the patient is determined by flow cytometry.

8. The method of claim 1, wherein the gamma-secretase inhibitor is selected from the group consisting of nirogacestat, crenigacestat, AL101, AL102, semagacestat, avagacestat, and ianabecestat, or pharmaceutically acceptable salt thereof.

9. The method of claim 1, wherein the gamma-secretase inhibitor is nirogacestat or pharmaceutically acceptable salt thereof.

10-20. (canceled)

21. A method for treating multiple myeloma in a patient in need thereof comprising administering to the patient an oral dosage of nirogacestat or a pharmaceutically acceptable salt thereof wherein the oral dosage provides a mean maximum drug plasma concentration (Cₘₐₓ) of more than 225 ng/ml.

22-23. (canceled)

24. A method for treating multiple myeloma in a patient in need thereof comprising administering of the patient an oral dosage of nirogacestat or a pharmaceutically acceptable salt thereof in combination with a B-cell maturation antigen (BCMA)-directed therapy to the patient.

25. The method of claim 24, wherein the BCMA-directed therapy is one or more of an allogenic chimeric antigen receptor T cell therapy, an autologous chimeric antigen receptor T cell therapy, an immunotherapy, an antibody drug conjugate therapy, or a bispecific antibody therapy9 with dual specificity for BCMA and an immune-related target.

26. A method for treating multiple myeloma in a patient in need thereof comprising administering to the patient an oral dosage of nirogacestat or a pharmaceutically acceptable salt thereof in combination with belantamab mafodotin.

27. The method of claim 8, wherein the nirogacestat, or pharmaceutically acceptable salt thereof, is nirogacestat hydrobromide.

28. The method of claim 27, wherein the nirogacestat hydrobromide is nirogacestat dihydrobomide.

29-39. (canceled)

40. The method of claim 1, wherein the patient is human.
